(19) United States
(12) Patent Application Publication
(10) Pub. No.: US 2013/0131487 A1
(43) Pub. Date: May 23, 2013

(54) TEST OBJECT INFORMATION ACQUISITION APPARATUS

(71) Applicant: CANON KABUSHIKI KAISHA, Tokyo (JP)

(72) Inventor: Daisuke Nagao, Kawaguchi-shi (JP)

(73) Assignee: CANON KABUSHIKI KAISHA, Tokyo (JP)

(21) Appl. No.: 13/662,003

(22) Filed: Oct. 26, 2012

(30) Foreign Application Priority Data
Oct. 31, 2011 (JP) 2011-239024

(51) Int. Cl. A61B 6/00

(52) U.S. Cl. 600/407

(57) ABSTRACT

An information acquisition apparatus configured to receive an elastic wave propagating through a test object to acquire characteristic information about the test object includes a receiver including an element configured to receive the elastic wave and to convert the received elastic wave into an electric signal, a time designation unit configured to designate a time required to acquire the characteristic information about the test object, a control unit configured to acquire area information about an area where the characteristic information about the test object is to be acquired based on the time and configured to cause a presentation unit to present the area information, and a scanning unit configured to cause the receiver to scan the test object based on the area information.

### BACKGROUND OF THE INVENTION

1. Field of the Invention

The present invention relates to a test object information acquisition apparatus, a method for controlling the apparatus, and a storage medium for causing a computer to perform the control method.

2. Description of the Related Art

An ultrasonograph (also called “sonograph”) is an apparatus for producing images obtained by ultrasonography, and has been known as a diagnostic apparatus for detecting deceases of human tissue, such as skin cancer and breast cancer. The ultrasonograph may receive an ultrasonic echo within a scanning range and obtain (or image) characteristic information about a test object within the scanning range by using an ultrasonic sending and receiving element to scan the test object. Thus, an area, which is larger in size than the ultrasonic sending and receiving element, can be imaged. For such a technique, Japanese Patent Application Laid-Open No. 2005-218520 discusses how a user previously designates an imaging area of a test object to image the designated imaging area. Japanese Patent Application Laid-Open No. 2005-218520 also discusses how a positional relationship between the designated imaging area and a maximum imageable area is displayed on a display unit, in consideration of the size of an ultrasonic sending and receiving element.

However, the ultrasonograph using the technique discussed in Japanese Patent Application Laid-Open No. 2005-218520 does not include a unit for calculating an imaging area from a time required to acquire an ultrasonic echo in imaging and a time of binding a subject. To accurately perform imaging (acquire characteristic information) in the photoacoustic imaging apparatus, the test object is preferably imaged in a resting state. To that end, the subject generally needs to be bound (e.g., to a gantry) using any method such as a method for keeping at least part of the body fixed. Particularly, when the breast of a subject is to be imaged, the beast is fixed in a compressed state; and this may be painful. The time elapsed from the start of imaging until the subject is released is referred to as a binding time. In a case where, the subject suffers discomfort or pain, the binding time should be minimized. Therefore, information about an estimated time elapsed from the start of imaging until the subject is released is useful to a doctor, an operator who performs imaging, or the subject. However, conventional apparatuses lack structure and functionally to provide information of binding time. This leads to a lack of convenience because the conventional ultrasonograph cannot indicate how large an area of a subject can actually be imaged within a predetermined time. On the other hand, in diagnoses of skin cancer and breast cancer, a photoacoustic tomograph (hereinafter also referred to as a photoacoustic imaging apparatus) has begun to be proposed in addition to the well-known ultrasonograph. When irradiating a body tissue with measurement light such as visible light or near-infrared light, the photoacoustic tomograph measures a photoacoustic wave, which is generated after a light-absorbing material within a living organism absorbs energy of the measurement light and expands instantaneously. In this manner, the photoacoustic tomograph can visualize information about the body tissue. A technique for photoacoustic imaging enables a distribution of absorption densities of light energy, i.e., a distribution of densities of the light-absorbing material within the living organism to be measured quantitatively or three-dimensionally. Thus, a photoacoustic imaging apparatus has significant advantages over its sonographic counter part. For example, the burden on patients is much lower because the photoacoustic apparatus, by using light to capture a diagnostic image, enables diagnostic imaging without any radiation exposures or invasive procedures. Therefore, the photoacoustic imaging apparatus is expected to be put into practical use for screening of breast cancer and early diagnosis of other tissue deceases instead of an X-ray apparatus, which cannot easily be used for repeated diagnosis imaging.

However, in the photoacoustic imaging apparatus, as discussed above, there has also been a desire to grasp how much the size of an area can be imaged within a predetermined time, similar to the above-mentioned ultrasonograph, because information about a test object within a scanning range may be obtained (imaged) by using an element configured to receive a photoacoustic wave to scan the test object.

### SUMMARY OF THE INVENTION

Embodiments of the present invention is directed to an information acquisition apparatus configured to acquire, from a test object, an elastic wave such as a photoacoustic wave with an ultrasonic probe, capable of presenting information about an imaging area, depending on a time required to acquire information about a test object, which has been designated by a user, or a time of binding a subject.

According to an aspect of the present invention, an information acquisition apparatus configured to receive an elastic wave propagating through a test object to acquire characteristic information about the test object includes a receiver including an element configured to receive the elastic wave and to convert the received elastic wave into an electric signal, a time designation unit configured to designate a time required to acquire the characteristic information about the test object, a control unit configured to acquire area information about an area where the characteristic information about the test object is to be acquired based on the time and configured to cause a presentation unit to present the area information, and a scanning unit configured to cause the receiver to scan the test object based on the area information.

According to an exemplary embodiment of the present invention, information about an area where test object information about a test object is acquirable can be presented when the test object information is acquired (imaged), so that conveniences for a user and a subject are improved.

Further features and aspects of the present invention will become apparent from the following detailed description of exemplary embodiments with reference to the attached drawings.

### BRIEF DESCRIPTION OF THE DRAWINGS

The accompanying drawings, which are incorporated in and constitute a part of the specification, illustrate exemplary embodiments, features, and aspects of the invention and, together with the description, serve to explain the principles of the invention.

FIG. 1 illustrates a configuration of a photoacoustic imaging apparatus according to a first exemplary embodiment of the present invention.

FIG. 2 illustrates a scanning locus of a probe when an imaging area is designated.

FIG. 3 is a flowchart illustrating a scanning locus of a probe within an imaging designation area.

FIG. 4 illustrates a scanning locus of a probe within an imaging designation area.

FIG. 5A is a flowchart illustrating scanning area calculation. FIG. 5B is a subroutine of FIG. 5A.

FIG. 6 illustrates an example of a setting screen of an imaging time.

FIG. 7 illustrates coordinates and a condition during scanning area calculation.

FIG. 8 illustrates a configuration of a photoacoustic imaging apparatus according to a second exemplary embodiment of the present invention.

FIG. 9 illustrates an example of an area that can be imaged within an imaging time.

### DESCRIPTION OF THE EMBODIMENTS

Various exemplary embodiments, features, and aspects of the invention will be described in detail below with reference to the drawings.

While a photoacoustic tomography (photoacoustic imaging apparatus) is described as an example of a test object information acquisition apparatus, an exemplary embodiment of the present invention is not limited to this, but is also applicable to an ultrasonograph. Further, in the photoacoustic tomography, the scope of the invention is not limited to an illustrated example.

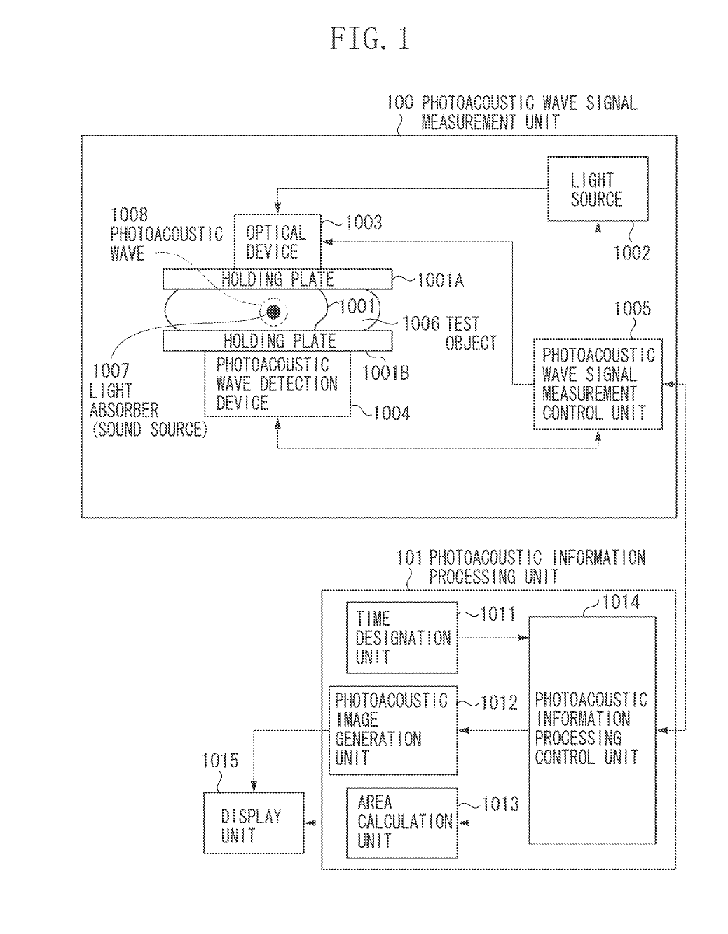

FIG. 1 illustrates the outline of a test object information acquisition apparatus according to a first exemplary embodiment of the present invention and a test object information acquisition system including the test object information acquisition apparatus and a presentation unit. A photoacoustic imaging apparatus serving as the test object information acquisition apparatus according to the present exemplary embodiment includes a photoacoustic wave signal measurement unit 100 including at least a photoacoustic wave detection device 1004 serving as a receiver including an element configured to receive a photoacoustic wave 1008 serving as an elastic wave propagating through a test object 1006 and convert the received photoacoustic wave 1008 into an electric signal, and a photoacoustic wave signal measurement control unit 1005 also serving as a scanning unit configured to cause the photoacoustic wave detection device 1004 serving as the receiver to scan the test object 1006, as illustrated in FIG. 1. The photoacoustic imaging apparatus also includes a photoacoustic information processing unit 101 including at least a time designation unit 1011 configured to designate a time required to acquire characteristic information about the test object 1006 and a photoacoustic information processing control unit 1014 constituting a control unit configured to acquire area information about an area where the characteristic information about the test object 1006 is to be acquired and to cause a display unit 1015 serving as the presentation unit to present the acquired area information based on the time designated by the time designation unit 1011.

The test object information acquisition apparatus according to the present exemplary embodiment having the above-mentioned configuration can previously confirm the size and the position of an imageable area based on a time designated by a user, while the photoacoustic wave detection device 1004 serving as the receiver scans the test object 1006 to acquire the characteristic information about the test object 1006 in the test object information acquisition apparatus. This results in an improvement in convenience for the user or a subject. More specifically, the test object information acquisition apparatus can previously grasp an imaging range, even though an imaging time and the imaging range differ for each imaging (measurement) in such a scanning-type apparatus, thereby improving convenience for the user and the subject. For example, the imaging time needs to be limited depending on an individual difference for each subject. More specifically, in a subject having weak physical strength (e.g., an aged person) or a subject having a low tolerance for pain with a test object thereof compressed during imaging, as described below, the imaging time needs to be limited (shortened). In such a case, the test object information acquisition apparatus according to the present exemplary embodiment can previously designate (limit) the imaging time and grasp a range that can be imaged within the limited imaging time. This results in an improvement in convenience for the user and the subject.

In the test object information acquisition apparatus according to the present exemplary embodiment, the photoacoustic wave signal measurement unit 100 further includes a holding plate 1001, a light source 1002, and an optical device 1003; and the photoacoustic information processing unit 101 further includes a photoacoustic image generation unit 1012, and an area calculation unit 1013, as illustrated in FIG. 1. The area calculation unit 1013, together with the photoacoustic information processing control unit 1014, constitutes the control unit. The test object information acquisition apparatus further includes the display unit 1015 serving as the presentation unit as the test object information acquisition system. Details of the photoacoustic wave signal measurement unit 100, the photoacoustic wave information processing unit 101, and the display unit 1015 will be described below. A configuration of the photoacoustic wave signal measurement unit 100 will be first described.

In FIG. 1, the test object 1006 to be imaged is fixed to the holding plate 1001 configured to compress and fix the test object 1006 from both sides. The holding plate 1001 constituting a holding unit includes a pair of holding plates 1001A and 1001B, and a holding mechanism (not illustrated) controls a holding position to change a holding clearance and holding pressure. The holding plates 1001A and 1001B are collectively referred to as the holding plate 1001 when they need not be distinguished. The holding plate 1001 fixes the test object 1006 to the test object information acquisition apparatus with the test object 1006 held (pressed) between the holding plates 1001A and 1001B. This can prevent a measurement error from occurring when the test object 1006 moves. In addition, the thickness of the test object 1006 can be adjusted to be suitable for photoacoustic wave measurement depending on the penetration depth of measurement light. The holding plate 1001 can include a contacting member (e.g. a film or gel in contact with the test object 1006) having a high transmittance of measurement light as well as having high acoustic alignment (resonance) with an ultrasonic probe (the measurement unit within the photoacoustic wave detection device 1004). This contacting member with high transmittance and predetermined acoustic resonance is advantageous because the holding plate 1001 is positioned in an optical path of the measurement light. Examples of the contacting member include polymethylpentene or other like polymer used in ultrasonography.

The light source 1002 irradiates the test object 1006 with light, to generate the photoacoustic wave 1008 serving as an elastic wave from the test object 1006. The light source 1002 includes two light sources (referred to as a light source A and a light source B in the following description), which are not illustrated. The light source 1002 generally uses a solid-state laser (e.g., a yttrium-aluminum-garnet laser or a titanium-sapphire laser) capable of emitting a pulse of light having a central wavelength in a near-infrared area. The wavelength of the measurement light (light with which the test object 1006 is irradiated) is selected in a range of 530 nm to 1300 nm depending on a light-absorbing material (e.g., hemoglobin, glucose, or cholesterol) within the test object 1006 to be imaged. For example, hemoglobin in a breast cancer new blood vessel to be imaged generally absorbs light having a wavelength of 600 nm to 1000 nm. On the other hand, a light absorber of water composing a living organism reaches its minimum when light has a wavelength in the vicinity of 830 nm so that the absorption of the light is relatively increased when the light has a wavelength of 750 nm to 850 nm. The rate of absorption of light changes depending on a state of hemoglobin (oxygen saturation). Therefore, a functional change of the living organism may be measurable by comparison of the changes. While two light sources have been used in the present exemplary embodiment, a single or three or more light sources may be used. The light source generally has a determined irradiation frequency. The irradiation frequency is determined as a designed value to continuously irradiate pulsed light having a desired intensity. The irradiation frequency is preferably to be high because the frequency affects the number of times of measurement of the photoacoustic wave 1008 transmitted per unit time. In the present exemplary embodiment, both of the two light sources A and B have a pulse frequency of 20 Hz.

When the optical device 1003 for irradiating the test object 1006 with the measurement light from the light source 1002 in a desired shape includes an optical system such as a lens, a mirror, or an optical fiber, and a scanning mechanism for scanning with respect to the holding plate 1001. Any optical system may be used as long as the test object 1006 is irradiated with the measurement light emitted from the light source 1002 in a desired shape.

When the test object 1006 is irradiated with the measurement light generated by the light source 1002 via the optical device 1003, a light absorber 1007 within the test object 1006 absorbs the light, and releases the photoacoustic wave 1008 serving as an elastic wave. In this case, the light absorber 1007 corresponds to a sound source.

The photoacoustic wave detection device 1004 serving as a light detector including an element configured to receive the photoacoustic wave 1008 serving as an elastic wave generated in the light absorber 1007 and to convert the detected photoacoustic wave 1008 into an electric signal detects the photoacoustic wave 1008, and converts the detected photoacoustic wave 1008 into an electric signal. The photoacoustic wave 1008 generated from the living organism is an ultrasonic wave having a frequency of 100 KHz to 100 MHz. Therefore, elements (receiving elements) capable of receiving the above-mentioned frequency band are used for the photoacoustic wave detection device 1004. Any elements, (receiving elements) such as a transducer using a piezoelectric phenomenon, a transducer using resonance of light, or a transducer using a change in capacitance, may be used, as long as they can detect the photoacoustic wave 1008 serving as an elastic wave. The photoacoustic wave detection device 1004 serving as the light detector according to the present exemplary embodiment has a plurality of receiving elements two-dimensionally arranged therein. Such elements are used in a two-dimensional arrangement so that the photoacoustic wave 1008 serving as an elastic wave can be detected simultaneously at a plurality of locations. In this manner, a detection time can be shortened, and an adverse effect due to vibration of the test object 1006 can be reduced. As an example, a receiver including 20 receiving elements arranged in a scanning direction and 20 receiving elements arranged in a sub-scanning direction at a pitch of 4 mm can be appropriately used. Details of the main scanning direction and the sub-scanning direction will be described below. In the present exemplary embodiment, the test object 1006 is irradiated with the measurement light from a surface directly opposite to (in front of) the photoacoustic wave detection device 1004 serving as the receiver. Therefore, the optical device 1003 is arranged opposite to the photoacoustic wave detection device 1004, and scanning control is simultaneously performed on the optical device 1003 and the photoacoustic wave detection device 1004 to keep a positional relationship therebetween.

The photoacoustic wave signal measurement control unit 1005 performs amplification processing for the electric signal based on the photoacoustic wave 1008 obtained from the photoacoustic wave detection device 1004 serving as the receiver, conversion processing from an analog signal to a digital signal, and integration processing for reducing noise. The photoacoustic wave signal measurement control unit 1005 sends a photoacoustic wave signal to an external device such as the photoacoustic wave information processing control unit 1014 via an interface (not illustrated).

The photoacoustic wave signal measurement control unit 1005 includes the scanning unit, and controls scanning of the test object 1006 with the optical device 1003 and the photoacoustic wave detection device 1004. The photoacoustic wave signal measurement control unit 1005 also controls driving of the light source 1002, the optical device 1003, and the photoacoustic wave detection device 1004. The foregoing will be described below in more detail.

The integration processing is performed to repeatedly measure one and the same portion of the test object 1006 and perform averaging processing to reduce a system noise, to improve a signal-to-noise (S/N) ratio of the photoacoustic wave signal 1008. While details of control of the scanning in the optical device 1003 and the photoacoustic wave detection device 1004 will be described below, the optical device 1003 and the photoacoustic wave detection device 1004 maybe caused to scan the test object 1006 in two dimensions and measure the test object 1006 at each scanning position. The photoacoustic wave signal measurement control unit 1005 including the scanning unit performs this scanning based on an area (where the characteristic information about the test object 1006 is to be acquired) calculated by the area calculation unit 1013 (details thereof will be described below) that constitutes the control unit together with the photoacoustic wave information processing control unit 1014. The photoacoustic wave detection unit 1004 serving as the receiver is thus caused to scan the test object 1006 so that the photoacoustic wave 1008 required in a wide imaging area can be acquired even with a small-sized probe. For example, in breast imaging, a photoacoustic image of an entire breast can be captured. The imaging area is an area where three-dimensional volume data calculated based on the measured photoacoustic wave 1008 is acquired.

Control of the light source 1002 includes selection of the light source A or the light source B, and irradiation timing of the laser. Control of the optical device 1003 and the photoacoustic wave detection device 1004 includes movement control (for movement to an appropriate position) relating to an incidental time, described below.

The photoacoustic wave information processing unit 101 will be described below. The photoacoustic wave information processing unit 101 designates the time required to acquire the characteristic information about the test object 1006, and calculates and acquires the area (also referred to as an imaging area, a measurement area, and a scanning area) where the characteristic information about the test object 1006 is to be acquired based on the designated time. In addition, the photoacoustic wave information processing unit 101, generates and displays a photoacoustic wave image based on the photoacoustic wave measurement data received from the photoacoustic wave signal measurement unit 100. Further, the photoacoustic wave information processing unit 101 performs processing, for example, for displaying information about the acquired area. The photoacoustic wave information processing unit 101 generally uses a device having a high-performance arithmetic processing function and a graphics display function, e.g., a personal computer or a work station equipped with appropriate hardware programmed software algorithms, as described below.

The time designation unit 1011 configured to designate the time required to acquire the characteristic information about the test object 1006 designates a time required to acquire the characteristic information using an interface device (an input unit), such as a mouse. The time required to acquire the characteristic information includes a time required to scan the test object 1006 and acquire (measure) the photoacoustic wave 1008 serving as an elastic wave and the incidental time. Details thereof will be described below. The input unit is not limited to a mouse or a keyboard. The input unit may be of a pen tablet type, or may be a touch pad attached to the surface of a display device.

The photoacoustic information processing control unit 1014 constituting the control unit receives information about the time required to acquire the characteristic information about the test object 1006, which has been obtained by the time designation unit 1011, calculates area information about the area where the characteristic information about the test object 1006 is to be acquired, along with the area calculation unit 1013, described below, and displays the calculated information on the display unit 1015 serving as the presentation unit.

The area calculation unit 1013, together with the photoacoustic information processing control unit 1014, constituting the control unit calculates information about an imaging area. Details thereof will be described below. The information about the imaging area, which has been calculated by the area calculation unit 1013, is displayed on the display unit 1015 serving as the presentation unit under an instruction from the area calculation unit 1013.

The photoacoustic imaging apparatus having the above-mentioned configuration acquires the characteristic information about the test object 1006 based on a photoacoustic effect so that a distribution of optical characteristics of the test object 1006 can be imaged and presented as a photoacoustic image. While the photoacoustic wave signal measurement unit 100 and the photoacoustic wave information processing unit 101 are constituted in separate types of hardware in FIG. 1, their respective functions may be collected and integrated.

A method for controlling the photoacoustic imaging apparatus serving as the information acquisition apparatus will be described below based on the above-mentioned configuration of the photoacoustic imaging apparatus.

A method for controlling the photoacoustic imaging apparatus (information acquisition apparatus), according to the present exemplary embodiment, includes the following processing operations:
Receiving information about a time required to acquire characteristic information about a test object, which has been designated by a user;
Acquiring area information about an area where the characteristic information about the test object is to be acquired based on a designated time;
Causing a presentation unit to present the acquired area information; and
Causing a receiver including an element configured to receive an elastic wave and convert the received elastic wave into an electric signal to scan the test object based on the acquired area information.

Each processing operation will be described in detail below after the outline of movement of the receiver during imaging and the breakdown of a time required for the imaging.

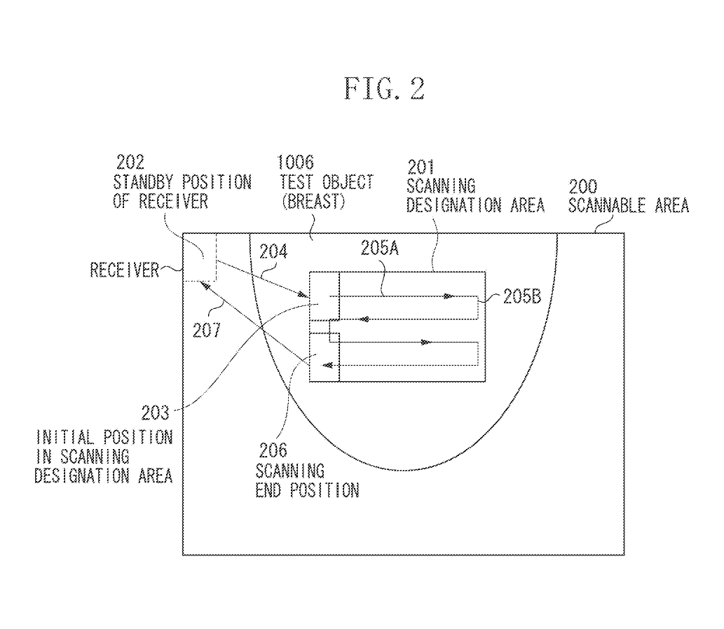

FIG. 2 is a conceptual diagram illustrating a scanning locus of the center of the photoacoustic wave detection device 1004 serving as the receiver when the receiver images a determined area.

A scannable area 200 represents a maximum area which can be scanned on a scanning surface; and a scanning designation area 201 represents an area that is scanned by the receiver, e.g., a scanning area on the scanning surface corresponding to an imaging area calculated from a time required to acquire the photoacoustic wave 1008 serving as an elastic wave. The calculation of the imaging area will be described below. The photoacoustic wave detection device 1004 (receiver) performs scanning by moving from a standby position 202 to an initial position 203 in the scanning designation area 201 (see an arrow 204 illustrated in FIG. 2). The photoacoustic wave detection device 1004 then scans the whole of the scanning designation area 201 in a main scanning direction 205A and a sub-scanning direction 205B, to measure the photoacoustic wave 1008, and then moves from a scanning end position 206 to the standby position 202 (see an arrow 207 illustrated in FIG. 2).

Details of scanning within the scanning designation area 201 in the photoacoustic wave detection device 1004 serving as the receiver will be described below.

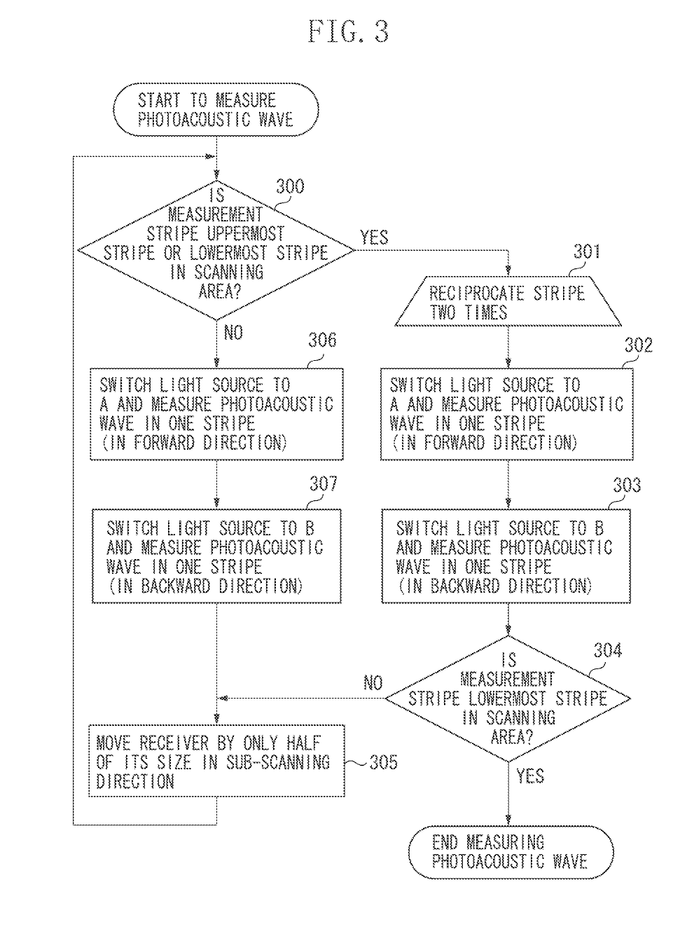

A flowchart illustrated in FIG. 3 represents the flow of photoacoustic wave measurement in the scanning designation area 201 illustrated in FIG. 2.

In the present exemplary embodiment, the number of times of integration of photoacoustic wave data per pixel is set to 40. In an example of the present exemplary embodiment, the number of elements constituting a probe is 20 in the main scanning direction 205A (shown in FIG. 2), and the number of times of integration is set to 40. Therefore, the probe is moved by an amount corresponding to one receiving element so that integration can be performed 20 times in a forward direction (and also 20 times in a backward direction).

An area where the photoacoustic wave 1008 is measured by moving the probe in the main scanning direction 205A is defined as a stripe. Particularly in the present exemplary embodiment, a size in which a photoacoustic wave signal can be acquired by emitting light from the light source 1002 once is the size of an area of all the elements constituting the probe. Actually, the area where the photoacoustic wave 1008 is measured is a three-dimensional area including a depth direction. However, a plane cutout in a plane parallel to scanning with the probe from the area where the photoacoustic wave 1008 is measured is referred to as a stripe, unless otherwise stated.

The flow of scanning (measurement) of a predetermined imaging area will be described with reference to the flowchart illustrated in FIG. 3.

After the photoacoustic wave detection device 1004 serving as the receiver moves to the initial position 203 in the scanning designation area 201, the process of photoacoustic wave measurement is started.

In step 300, the photoacoustic wave detection device 1004 determines whether the subsequent measurement stripe is an uppermost stripe or a lowermost stripe in the scanning designation area 201, i.e., the first stripe or the last stripe in the measurement.

If the measurement stripe is the uppermost stripe or the lowermost stripe (YES in step 300), then in step 301, the photoacoustic wave detection device 1004 reciprocates in the measurement stripe two times. In step 302, the photoacoustic wave detection device 1004 switches the light source 1002 to the light source A and measures the photoacoustic wave 1008 in one stripe (in a backward direction). In step 303, the photoacoustic wave detection device 1004 switches the light source 1002 to the light source B and measures the photoacoustic wave 1008 in one stripe (in a backward direction). The reciprocation is performed two times because the number of times of integration is 40 under both the light source A and the light source B and at the same time, the number of times of integration in one stripe is 20.

In step 304, the photoacoustic wave detection device 1004 then determines whether the measurement stripe is the lowermost stripe in the scanning designation area 201. If the measurement stripe is the lowermost stripe (YES in step 304), the photoacoustic wave measurement in the scanning area, which has been calculated from the time, ends. If the measurement stripe is not the lowermost stripe (NO in step 304), then in step 305, the photoacoustic wave detection device 1004 serving as the receiver moves by only half of its size in the sub-scanning direction 205B.

If the measurement stripe is neither the uppermost stripe nor the lowermost stripe (NO in step 300), then in step 306, the photoacoustic wave detection device 1004 serving as the receiver switches the light source 1002 to the light source A and measures the photoacoustic wave 1008 in one stripe (in a forward direction). In step 307, the photoacoustic wave detection device 1004 then switches the light source 1002 to the light source B and measures the photoacoustic wave 1008 in one stripe (in a backward direction). In step 305, the photoacoustic wave detection device 1004 is moved by only half of the size of the probe in the sub-scanning direction 205B. The photoacoustic wave detection device 1004 is moved by only half of its size for each stripe. Therefore, the number of times of integration reaches 40 under both the light source A and the light source B in one-time reciprocation in strips other than the uppermost stripe or the lowermost stripe.

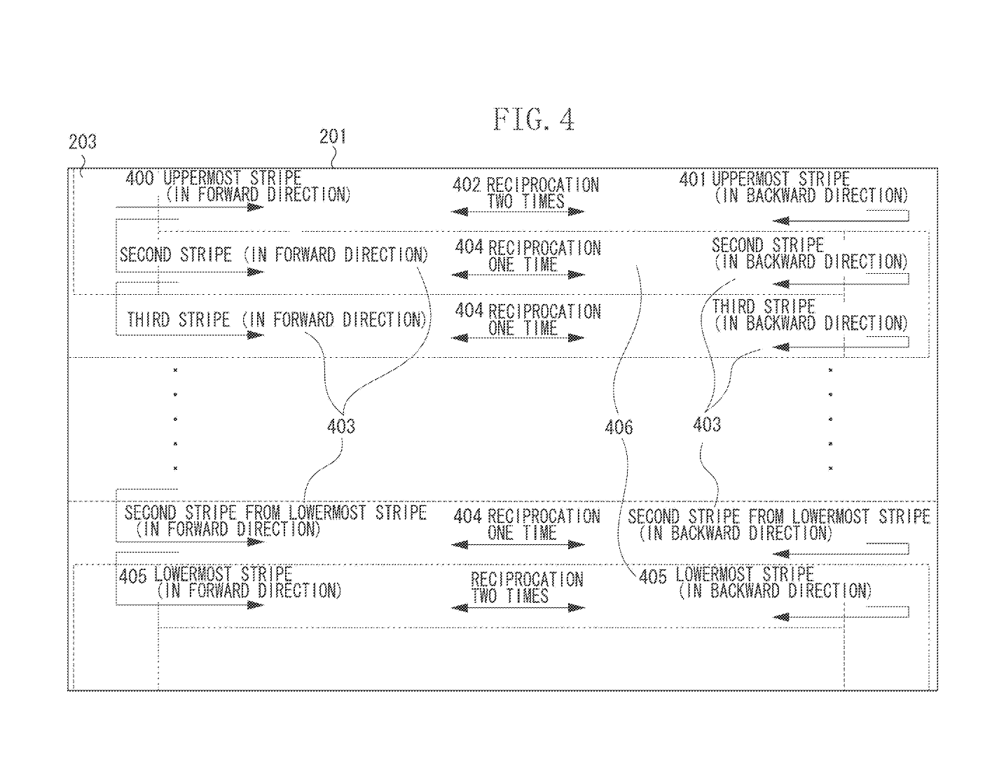

The above-mentioned scanning locus will be conceptually described in detail below with reference to FIG. 4. The photoacoustic wave 1008 is measured under the light source A in a forward direction 400 of the uppermost stripe from the initial position 203 in the scanning designation area 201, and is measured under the light source B in a backward direction 401 of the uppermost stripe. Thus, in the uppermost stripe, reciprocation for two times 402 is performed. The photoacoustic wave 1008 is then measured under the light source A and the light source B, respectively, in a forward direction and a backward direction of each of the second stripe 403 to the second stripe from the lowermost stripe 403. Thus, in the stripe 403, reciprocation for one time 404 is performed. For a lowermost stripe 405, measurement similar to that of the above-mentioned uppermost stripe is performed. Thus, in the lowermost stripe 405, reciprocation is performed for two times. The number of times of integration in the lower half of the uppermost stripe or at least the upper half of the lowermost stripe exceeds 40. However, this leads to an improvement in an S/N ratio and presents no problem. In an area 406, the number of times of integration exceeds 40.

To accurately perform imaging (acquire characteristic information) in the photoacoustic imaging apparatus, the test object 1006 is preferably imaged in a resting state. Accordingly, the subject generally needs to be bound using any method such as a method for keeping a part of the body thereof fixed. Particularly when the breast of the subject is imaged, the beast is fixed in a compressed state, to bind the subject. In such a case, the subject is bound often with pain. Therefore, information about an imaging area, which is calculated from a time elapsed from the start of imaging until the subject is released, is useful for a doctor and an operator who perform imaging and also for the subject. Even when the breast is not compressed, the subject should be bound because the test object 1006 needs to be imaged in a resting state. The time elapsed from the start of imaging until the subject is released is referred to as a binding time. The binding time will be divided into a time required for the receiver to scan and an incidental time in a description below.

The scanning time is a time required for the receiver to scan the designated imaging area, and the incidental time is a time required for the receiver to move between the standby position 202 and the designated imaging area and a time required to release the test object 1006, described below. This will be specifically described in a series of operations (1) to (4):

(1) A movement time T1 required to simply move from the standby position 202 of the photoacoustic wave detection device 1004 serving as the receiver to the initial position 203 in the scanning designation area 201.

(2) A movement time T2 required to scan the scanning designation area 201 in the main scanning direction 205A while acquiring the photoacoustic wave 1008.

(3) A movement time T3 required to simply scan the scanning designation area 201 in the sub-scanning direction 205B.

(4) A movement time T4 required to simply move from the scanning end position 206 to the standby position 202 of the photoacoustic wave detection device 1004 serving as the receiver.

In each of the foregoing operations (1) to (4), the scanning time is the sum of the movement times T2 and T3, and the incidental time is the sum of the movement times T1 and T4.

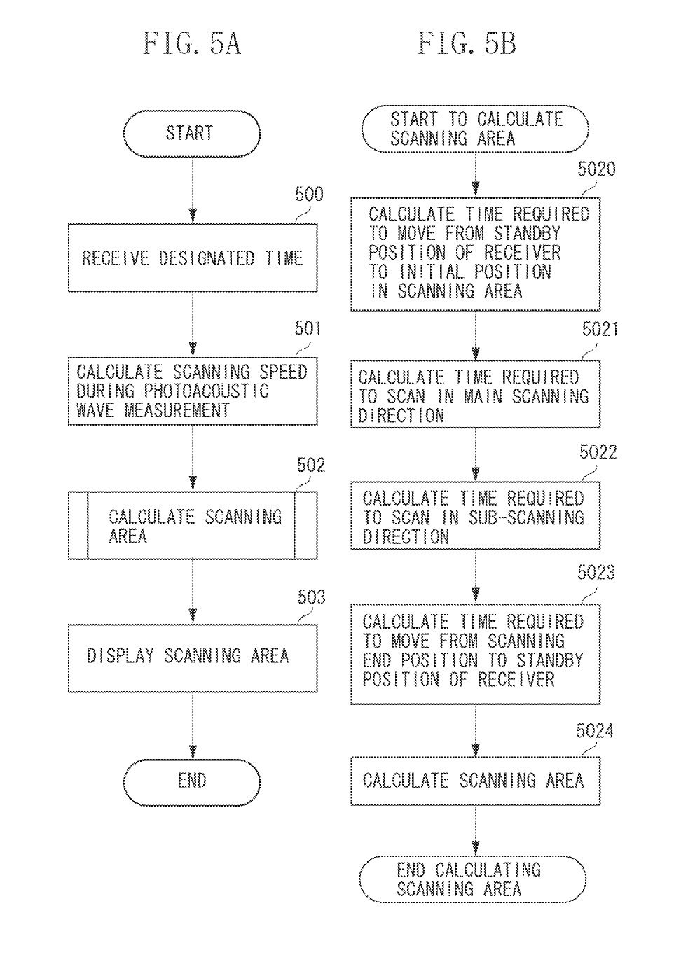

A method for controlling the test object information acquisition apparatus will be described with reference to FIGS. 5A and 5B based on the above-mentioned configuration of the photoacoustic imaging apparatus.

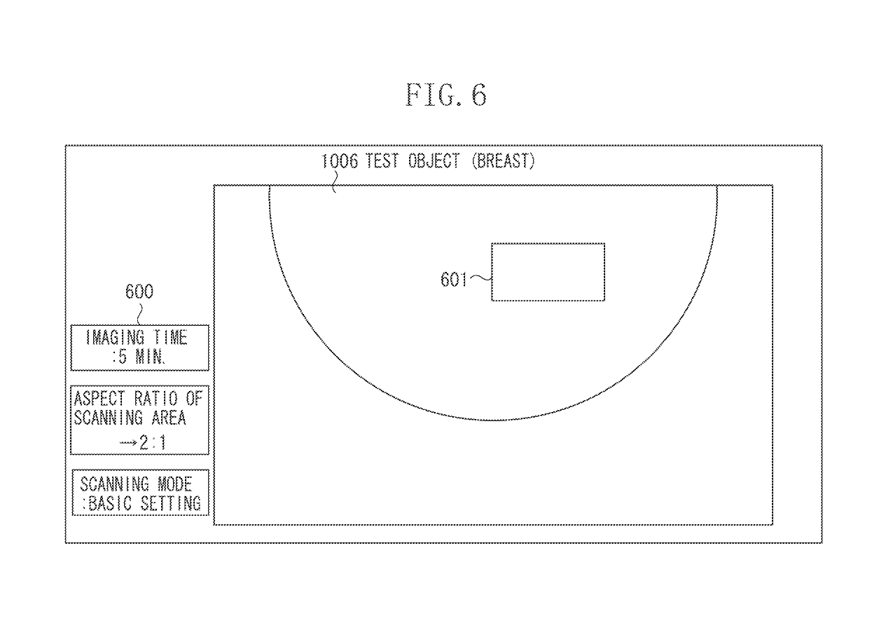

In FIG. 5A, at step 500, when a user designates a time to acquire the characteristic information about the test object 1006 by the time designation unit 1011, the photoacoustic information processing control unit 1014 receives the designated time. An input unit in the time designation unit 1011 is not limited to a mouse or a keyboard. For example, various input units such as an input unit of a tablet type and a touch pad attached to the surface of the display device can be used. An example of time designation is illustrated in FIG. 6. The user designates an imaging time 600. At this time, a measurement condition for photoacoustic wave measurement can also be set.

An area where the characteristic information is to be acquired is an area where the photoacoustic wave 1008 can be acquired within the time designated by the user. Thus, an area where to scan by the receiver is determined.

To calculate the scanning area serving as the area where the characteristic information is to be acquired, the following parameters are required. In the example of the present exemplary embodiment, the parameters are set as follows. For example, the speed of the probe during simple movement may be a non-constant value, considering an initial acceleration or the like, the shape of the scanning area may be a rhombus, and the scanning locus of the probe may draw a spiral shape. Each of the parameters may be settable by the user.

Speed of probe during simple movement: Vxy

Shape of scanning area: rectangle (including square)

Aspect ratio of scanning area: length:width=1:n

Scanning locus of probe: as described in the description about the scanning locus in the scanning designation area

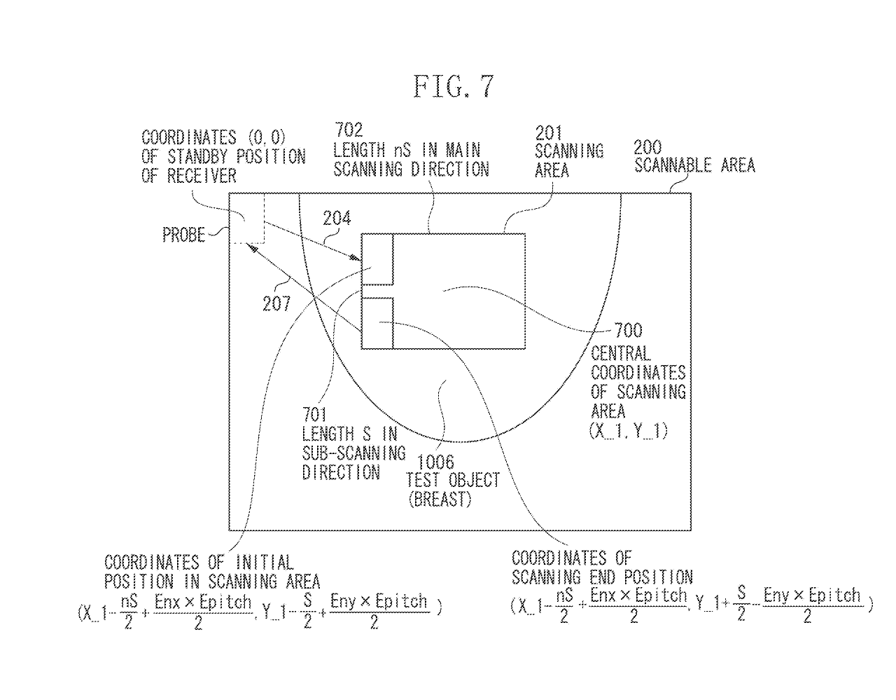

Central coordinates of scanning area: (X_1, Y_1) (700 in FIG. 7)

Scanning time: details will be described below

Movement speed of probe during photoacoustic wave acquisition: details will be described below.
However, the scope of the present invention is not limited to the above parameter settings.

In step 501, the photoacoustic information processing control unit 1014 calculates a scanning speed during the photoacoustic wave measurement. The number of elements in the main scanning direction 205A of the photoacoustic wave detection device 1004 serving as the receiver is set to Enx (elements), the number of elements in the sub-scanning direction 205B is set to Eny, a pitch between the elements is set to Epitch (mm), the number of times of integration in the photoacoustic wave measurement is set to Mn (times), and the light emission frequency of the light source 1002 is set to LHz (Hz). To simplify the description, if the number of times of integration Mn is a multiple of the number of elements Enx, a scanning speed Vx (mm/sec) and the number of times of scanning St (times) in the main scanning direction 205A of the photoacoustic wave detection device 1004 serving as the receiver and the light source 1002 are calculated by the following equations (1) and (2):

Vx=Epitch×LHz (1)

St=(Mn/Enx)×2×(½) (2)

In the example of the present exemplary embodiment, the number of elements constituting the probe is set to 20 in the main scanning direction 205A, and the number of times of integration is set to 40, as described above. Therefore, the photoacoustic wave detection device 1004 serving as the receiver is moved by an amount corresponding to one receiving element so that integration can be performed 40 times in one-time reciprocation.

Therefore, if a pitch of elements in one-time reciprocation is set to 4 mm, and the pulse rate (or frequency of emission) of the light source 1002 is set to 20 Hz, the scanning speed during the measurement is to be 80 mm/sec.

A speed calculation unit in the photoacoustic information processing control unit 1014 constituting the control unit calculates the scanning speed based on the foregoing description, i.e., an arrangement pitch of a plurality of elements arranged in a direction of scanning and the light emission frequency of the light source 1002. A scanning time is calculated and acquired based on a calculation result by the speed calculation unit.

Under a more complex condition, if the number of times of integration is smaller than the number of elements Enx in the main scanning direction 205A or is a multiple of a value smaller than Enx, the number of times of integration per reciprocation in movement of the photoacoustic wave detection device 1004 serving as the receiver is reduced. In this case, the photoacoustic wave detection device 1004 serving as the receiver can scan the test object 1006 while shifting by two pixels or more per unit time. Therefore, the scanning speed is set to be high. The movement speed of the photoacoustic wave detection device 1004 serving as the receiver is not limited to one in the method described in the example of the present exemplary embodiment. The movement speed may depend on a measurement condition and a device configuration. Various algorithms are expected to be applied to adjust the scanning speed.

The object of a scanning speed calculation function in the present exemplary embodiment is to find the movement speed of the photoacoustic wave detection device 1004 serving as the receiver for the photoacoustic wave measurement. Therefore, reference parameters and algorithms are not limited to those in a mode described in the above-mentioned example.

In step 502, the photoacoustic information processing control unit 1014 calculates the scanning area. In the example of the present exemplary embodiment, when the coordinates of a standby position 202 of the receiver (the photoacoustic wave detection device 1004) are (0, 0), and the length 701 in a sub-scanning direction of the scanning area 201 is S as illustrated in FIG. 7, the length 702 in a main scanning direction of a scanning area 201 is nS, and the coordinates of an initial position 203 in the scanning area 201 areas follows:

$$( X\_ 1 - \frac{nS}{2} + \frac{Enx \times Epitch}{2} , Y\_ 1 - \frac{S}{2} + \frac{Eny \times Epitch}{2} )$$

The coordinates of a scanning end position 206 are as follows:

$$( X\_ 1 - \frac{nS}{2} + \frac{Enx \times Epitch}{2} , Y\_ 1 + \frac{S}{2} - \frac{Eny \times Epitch}{2} )$$

A user may be able to designate the central coordinates of the scanning area 201 and the aspect ratio of the scanning area 201.

The process of step 502 is described in reference to

FIG. 5B. In step 5020, the photoacoustic information processing control unit 1014 calculates a movement time T1 required to move to the initial position 203 in the scanning area 201 as an incidental time.

The movement time T1 required to move to the initial position 203 in the scanning area 201 is expressed by the following equation (3):

$$T 4 = \frac{\sqrt{{( X\_ 1 - \frac{nS}{2} + \frac{Enx \times Epitch}{2} )}^{2} + {( Y\_ 1 + \frac{S}{2} - \frac{Eny \times Epitch}{2} )}^{2}}}{Vxy} \qquad ( 3 )$$

In step 5021, the photoacoustic information processing control unit 1014 calculates a movement time T2 required to scan in the main scanning direction. The number of stripes N covering the scanning area 201 when a movement distance in the sub-scanning direction is one-half of the size of the photoacoustic wave detection device 1004 serving as the receiver is expressed by the following equation (4). The calculated number of strips N represents the number of times the probe moves from an end to an end in the main scanning direction of the scanning area 201:

$$N = ceil ( \frac{\frac{S}{Eny \times Epitch}}{2} ) \qquad ( 4 )$$

Accordingly, the total movement distance in the main scanning direction in the scanning area 201 is calculated by nS×(N+1)×St. Therefore, the movement time T2 required to scan in the main scanning direction is expressed by the following equation (5):

$$T 2 = \frac{nS \times ( N + 1 ) \times St}{Vx} \qquad ( 5 )$$

The scanning speed Vx in the main scanning direction is 80 mm/sec in the above-mentioned example.

In step 5022, the photoacoustic information processing control unit 1014 calculates a movement time T3 required to scan in the sub-scanning direction. The movement time T3 in the sub-scanning direction is expressed by the following equation:

$$T 3 = \frac{S}{Vxy} \qquad ( 6 )$$

In step 5023, the photoacoustic information processing control unit 1014 calculates a movement time T4 required to move to the initial position 203 of the receiver (the photoacoustic wave detection device 1004) as an incidental time. The movement time T4 is expressed by the following equation (7):

$$T 4 = \frac{\sqrt{{( X\_ 1 - \frac{nS}{2} + \frac{Enx \times Epitch}{2} )}^{2} + {( Y\_ 1 + \frac{S}{2} - \frac{Eny \times Epitch}{2} )}^{2}}}{Vxy} \qquad ( 7 )$$

In step 5024, the photoacoustic information processing control unit 1014 calculates the scanning area 201. A scanning time T5 is expressed by the following equation (8):

T5=T2+T3 (8)

An incidental time T6 is expressed by the following equation (9):

T6=T1+T4 (9)

The sum of the scanning time T5 and the incidental time T6 is also the time received in step 500. The scanning area 201 can be calculated by solving the foregoing equation for the length Sin the sub-scanning direction. If the test object 1006 is held or compressed by the holding plate, a time required to release the test object 1006 from the holding plate 1001 may be included in the incidental time T6, as in the above-mentioned exemplary embodiment.

Referring back to FIG. 5A, in step 503, the photoacoustic information control unit 1014 converts the calculated scanning area 201 from an apparatus coordinate system to a camera coordinate system, and displays the scanning area 201 on a display unit. The scanning area may be surrounded with a frame 601 as illustrated in FIG. 6, may be filled in, or may be converted into an imaging area corresponding to the scanning area 201 when displayed.

While the scanning area 201 is calculated and displayed using the sum of the scanning time of the receiver and the movement time between the standby position 202 of the receiver and the imaging area as the time required to acquire the characteristic information in the present exemplary embodiment, the imaging area (where the characteristic information is to be acquired) may be further calculated and displayed based on a time required to bind the subject after the start of imaging and a time required to release the subject.

A program for causing a computer to perform the above-mentioned control method may also be included in the category of the exemplary embodiment of the present invention.

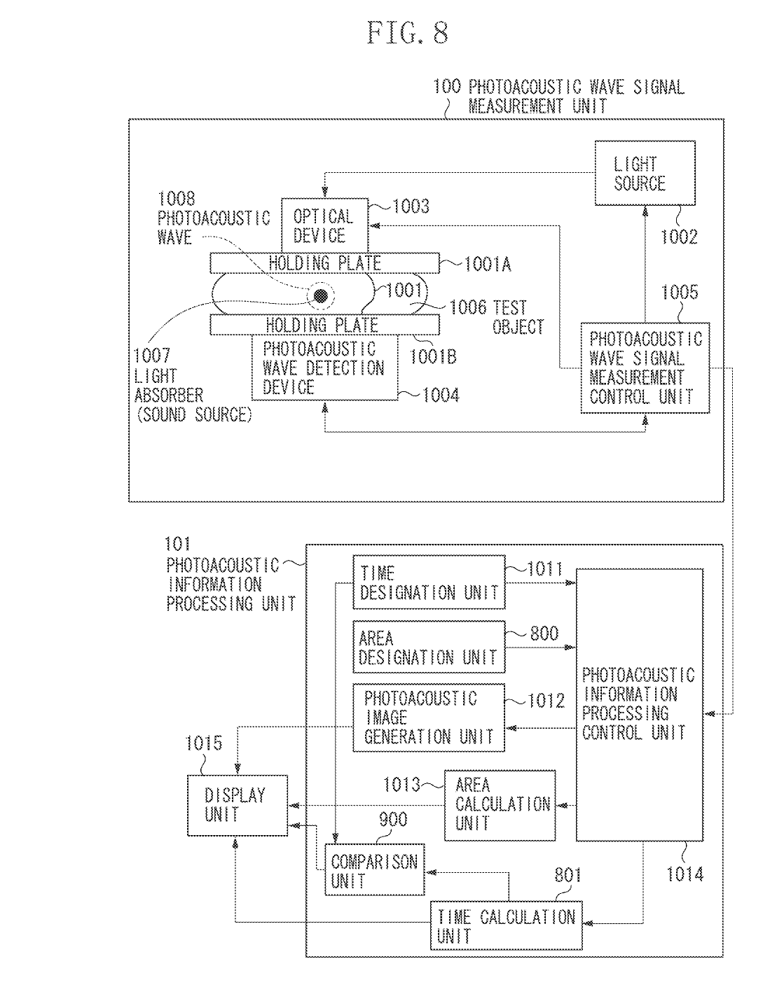

In a photoacoustic imaging apparatus according to a second exemplary embodiment of the present invention, an imaging area designation unit 800, a time calculation unit 801, and a comparison unit 900, which are added as constituent units will be described with reference to FIG. 8. The imaging area designation unit 800 includes a unit configured for a user to designate an imaging area. The imaging area is designated using an input unit such as a mouse. The input unit is not limited to a mouse or a keyboard. The input unit maybe of a pen tablet type, or may be a touch pad attached to a surface of a display device. The imaging area can be designated based on an image captured by a camera (not illustrated) installed in a direction perpendicular to a holding plate 1001A configured to compress and hold a test object 1006. The time calculation unit 801 calculates a time of binding a subject based on the imaging area designated by the imaging area designation unit 800. The time of binding the subject, which has been calculated by the time calculation unit 801, is displayed on a display unit 1015.

Processing for calculating and displaying a time required to acquire characteristic information based on the designated imaging area input from the imaging area designation unit 800 will be described.

The time required to acquire the characteristic information can be calculated when a photoacoustic information processing control unit 1014 constituting an information control unit relating to the imaging area input from the imaging area designation area 800 receives the total time of movement times (1) to (4) described in the first exemplary embodiment.

The calculated time is displayed on the display unit 1015. The time may be represented by a character such as a numeric character, a gauge, or an hour, or may be transmitted by a voice or the like. The time required to acquire the characteristic information maybe calculated not only based on a movement time and a scanning time of the receiver but also with the inclusion of a time required to bind the test object 1006 and a time required to release the test object 1006.

The comparison unit 900 compares a time designated by a time designation unit 1011 and the time calculated by the time calculation unit 801.

Processing for displaying an area where the characteristic information is to be acquired, which has been calculated by an area calculation unit 1013, based on a comparison result by the comparison unit 900 will be described below.

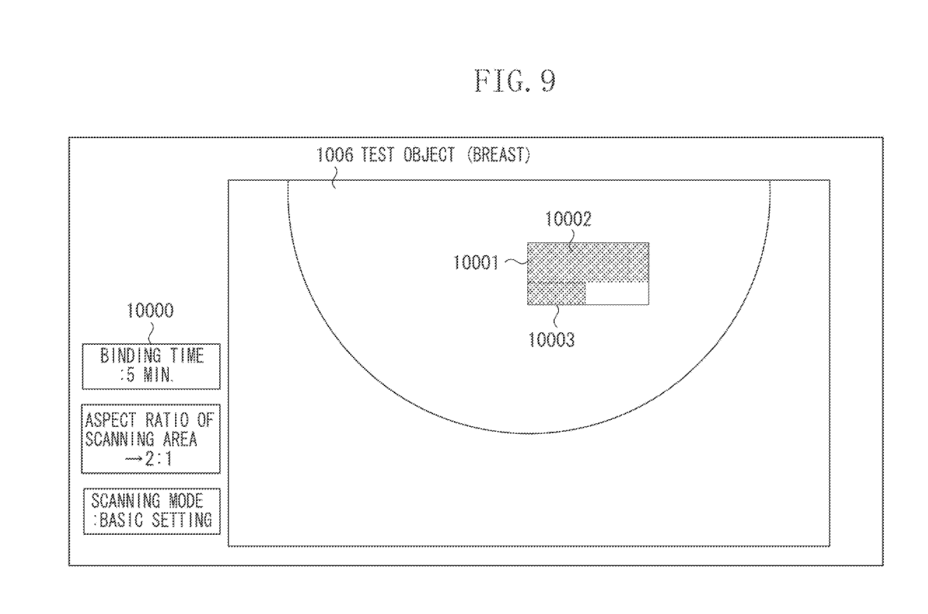

The photoacoustic information processing control unit 1014 receives a time 10000 required for the user to acquire the characteristic information, which has been designated by the time designation unit 1011, and a designated imaging area 10001, which has been designated by the area designation unit 800. The comparison unit 900 performs comparison to determine whether a time required to image the received designated imaging area 10001 is within the received time 10000 required to acquire the calculation information. As a comparison result, as illustrated in FIG. 9, an imageable area 10002 (where the characteristic information is to be acquired) in the received designated imaging area, within the received time 10000 required to acquire the characteristic information, may be filled in, surrounded with a frame, or displayed by OK or NG when displayed. If the area where the characteristic information is to be acquired (the area calculated by the area calculation unit 1013 from the time designated by the time designation unit 1011) is in a shape not suited to handle three-dimensional volume data of a photoacoustic wave 1008, the area maybe rounded. For example, an area 10003 maybe rounded to a rectangular parallelepiped shape. Further, if the imaging area is rounded, a time required to acquire the photoacoustic wave 1008 in the rounded imaging area may be displayed.

According to the second exemplary embodiment, a relationship between an imaging area and a binding time, which is useful information for a doctor and an operator who perform imaging.

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures, and functions.

This application claims priority from Japanese Patent Application No. 2011-239024 filed Oct. 31, 2011, which is hereby incorporated by reference herein in its entirety.

What is claimed is:

1. An information acquisition apparatus configured to receive an elastic wave propagating through a test object to acquire characteristic information about the test object, the test object information acquisition apparatus comprising:
a receiver including an element configured to receive the elastic wave and to convert the received elastic wave into an electric signal;
a time designation unit configured to designate a time required to acquire the characteristic information about the test object;
a control unit configured to acquire area information about an area where the characteristic information about the test object is to be acquired based on the time and configured to cause a presentation unit to present the area information; and
a scanning unit configured to cause the receiver to scan the test object based on the area information.

2. The information acquisition apparatus according to claim 1, wherein the control unit includes a speed calculation unit configured to calculate a scanning speed in causing the receiver to scan the test object, and
wherein, based on a calculation result by the speed calculation unit, the control unit is configured to acquire the area information about the area where the characteristic information is to be acquired.

3. The information acquisition apparatus according to claim 2, further comprising:
a light source configured to irradiate the test object with light, to generate the elastic wave from the test object,
wherein the receiver includes a plurality of the elements arranged in a direction of the scanning, and
wherein the speed calculation unit is configured to calculate the scanning speed based on a light emission frequency of the light source and an arrangement pitch of the elements.

4. The information acquisition apparatus according to claim 2, wherein the control unit is configured to acquire the area information about the area where the characteristic information is to be acquired, based on a time obtained by subtracting an incidental time from the time designated by the time designation unit and the scanning speed.

5. The information acquisition apparatus according to claim 4, wherein the incidental time includes one of a time required to bind the test object, a time required to release the test object, and a time required for the receiver to move between a standby position and the area.

6. The information acquisition apparatus according to claim 1, further comprising
an imaging area designation unit configured to designate an imaging area, and
a comparison unit configured to compare the imaging area designated by the imaging area designation unit and the area information about the area where the characteristic information is to be acquired,
wherein the control unit is configured to cause the presentation unit to present a comparison result by the comparison unit.

7. The information acquisition apparatus according to claim 1, further comprising:
an imaging area designation unit configured to designate an imaging area,
a time calculation unit configured to calculate an imaging time based on the imaging area designated by the imaging area designation unit, and
a comparison unit configured to compare the imaging time calculated by the time calculation unit and the time designated by the time designation unit,
wherein the control unit is configured to cause the presentation unit to present a comparison result by the comparison unit.

8. The information acquisition apparatus according to claim 1, wherein the presentation unit includes a display unit.

9. An information acquisition system comprising:
the information acquisition apparatus according to claim 1; and
the presentation unit,
wherein the presentation unit is configured to present the area information about the area where the characteristic information about the test object is to be acquired, by an instruction from the control unit.

10. A method for controlling an information acquisition apparatus configured to receive an elastic wave propagating through a test object to acquire characteristic information about the test object, the method comprising:
receiving information about a time required to acquire the characteristic information about the test object, which has been designated by a user;
acquiring area information about an area where the characteristic information about the test object is to be acquired based on the designated time;
causing a presentation unit to present the acquired area information; and
causing a receiver including an element, which is configured to receive the elastic wave and to convert the received elastic wave into an electric signal, to scan the test object based on the acquired area information.

11. A computer-readable storage medium storing a program for causing a computer to perform the method according to claim 10.
